(19) United States
(12) Patent Application Publication
(10) Pub. No.: US 2012/0226365 A1
(43) Pub. Date: Sep. 6, 2012

(54) METHOD AND DEVICE FOR CONTROLLING AN ARTIFICIAL ORTHOTIC OR PROSTHETIC JOINT

(75) Inventors: Martin Seyr, Wien (AT); Philipp Kampas, Wien (AT); Constantin Pop, Ternitz (AT)

(73) Assignee: OTTO BOCK HEALTHCARE PRODUCTS GMBH, Wien (AT)

(21) Appl. No.: 13/508,525

(22) PCT Filed: Nov. 12, 2010
(86) PCT No.: PCT/EP2010/006895

§ 371 Date: May 7, 2012

(30) Foreign Application Priority Data
Nov. 13, 2009 (DE) 102009052893.8

(51) Int. Cl. A61F 2/48

(52) U.S. Cl. 623/24

(57) ABSTRACT

The invention relates to a method and device for controlling an artificial orthotic or prosthetic joint with a resistance device to which at least one actuator is associated, via which the bending and/or stretching resistance is changed depending on sensor data. According to the invention, the resistance is adjusted depending on at least one measured temperature signal.

The invention relates to a method and an appliance for controlling an artificial orthotic or prosthetic joint with a resistance device to which at least one actuator is assigned, via which actuator the flexion and/or extension resistance is changed depending on sensor data. Such a method and such an appliance are suitable in particular for controlling joints of the lower extremities, that is to say the hip joint, the knee joint or the ankle joint. However, it is also possible in principle to use such an appliance and such a method to operate other prosthetic or orthotic artificial joints, for example an elbow joint or a joint in a prosthetic hand.

Artificial joints with resistance devices are increasingly being provided, which can be changed in terms of their flexion resistance or extension resistance. The ability so change the resistances has the advantage that it is possible to adapt the resistance to the different requirements during a sequence of movement. For example, in order to ensure that the different requirements during the various phases of a step are satisfied or supported in a way that is as natural as possible, controllable resistance devices are made available that provide an adapted, modifiable flexion resistance and extension resistance. By means of the flexion resistance, it is possible to establish how easily the lower leg socket or the lower leg rail swings back relative to the thigh socket or the thigh rail when a force is applied. The extension resistance brakes the forward movement, of the lower leg socket or of the lower leg rail and forms, among other things, an extension limit stop.

DE 10 2008 008 28 A1 discloses an orthopedic knee joint with an upper part and, arranged pivotably on the latter, a lower part which is assigned several sensors, for example a flexion angle sensor, an acceleration sensor, an inclination sensor and/or a force sensor. The extension limit stop is adjusted according to sensor data that are determined.

DE 10 2006 021 802 A1 describes a control system of a passive prosthetic knee joint with adjustable damping in the direction of flexion, for adaptation of a prosthetic device having upper attachment means and a connector element to an artificial foot. The adaptation is made to climbing stairs, wherein a low-moment lifting of the prosthetic foot is detected, and the flexion damping in the lifting phase is lowered to below a level that is suitable for walking on the flat. The flexion damping can be increased depending on the change in the knee angle and depending on the axial force acting on the lower leg.

DE 10 2007 053 389 A1 describes a method for controlling an orthopedic joint of a lower extremity with at least one degree of freedom, having an adjustable actuator by which an orthopedic device, comprising upper means of attachment to a limb and an orthopedic element arranged in an articulated manner distally from the attachment means, is adapted to a situation that deviates from walking on the flat. Several parameters of the orthopedic device are detected via sensors, the detected parameters are compared with criteria that have been established on the basis of several parameters and/or parameter profiles and are stored in a computer unit, and a criterion is then selected that is suitable on the basis of the detected parameters and/or parameter profiles. The movement resistances, movement ranges, drive forces or the profiles thereof are adapted, in accordance with the selected criterion, in order to control special functions that deviate from walking on the fiat.

U.S. R739,961 E describes a computer-controlled hydraulic resistance device for a prosthesis with pressure sensors. The pressure is detected, both on the high-pressure side and also on the low-pressure side, via a spring-biased magnet and a magnetically actuated, electronic sensor and is processed by a micro-controller in a closed loop in order to compensate automatically for variations in the appliance and in the viscosity of the hydraulic fluid. The results of the pressure measurement are based on the compensation.

As regards artificial knee joint prostheses or orthoses, it has proven useful that the joints offer a high degree of flexion resistance in the stance phase during walking or also during standing, wherein the joint is not completely locked. In this case, the slow bending of the joint during standing is prevented by the fact that the force vector lies in front of the joint axis and thus forces the joint to the extension limit stop. Whether the force vector lies in front of the joint axis is determined by the set-up of the prosthesis or orthosis.

The fact that the joint does not completely lock in the situation described above has the advantage that the user still has possible ways of intervening in the joint movement. For example, should he be standing on stairs and lose his balance, a locked joint would cause him to fall without any control, whereas he is still able to bend a joint with high resistance by means of the stump force. He can thus minimize the consequences of a fall or avoid falling altogether. The high flexion resistance also makes it easier to maneuver the joint in confined spaces or to sit down. A great benefit of the high flexion resistance is also that ramps and stairs can be descended alternatingly. The resistance of the joint takes over the support of the body and thus relieves the contralateral limb, that is to say the intact leg. This relief on the intact side is reflected directly in the energy to be dissipated by the resistance device, which energy can lead to very strong heating of the resistance device. This heating can also occur when walking on the flat, when the resistance device brakes the swing-out of the leg in the swing phase to a desired level, or when the wearer bends and extends the joint against a high resistance during the stance phase. In principle, heating of the resistance device can also occur in other joints, for example in the ankle joint or in the hip joint, or also in other joint devices, for example when high loads occur there.

The increases in temperature can lead to a change in resistance behavior and in some case to damage of the components of the joint device.

The object of the invention is to make available an appliance and a method by which these problems are reduced or eliminated.

According to the invention, this object is achieved by a method having the features of the main claim and by an appliance having the features of the additional independent claim. Advantageous embodiments and developments are set forth in the dependent claims.

In the method according to the invention for controlling an artificial orthotic or prosthetic joint with a resistance device to which at least one actuator is assigned, via which actuator the flexion and/or extension resistance is changed depending on sensor data, provision is made that the resistance is changed depending on a measured temperature or on a measured temperature signal. In this way, it is possible to protect the resistance device, or also other components of the artificial orthotic or prosthetic joint, against overheating. Heating can go so far as to cause the joint, to fail, because parts of the joint lose their shape or structural strength, or because the electronics are operated outside the permitted operating parameters. The resistance is preferably changed such that the dissipated energy is reduced. On account of the lower amount of energy to be converted, the resistance device or other components of the artificial joint can cool down and operate in a temperature range for which they are provided. In addition, provision can be made that the resistance device is adapted such that changes occurring as a result of a change of temperature are compensated. For example, if the viscosity of a hydraulic fluid decreases because of the heating, the resistance device can be suitably adjusted so as to continue to supply the usual flexion resistances and extension resistances, such that the prosthesis user or orthosis user can continue to rely on the known behavior of the artificial joint.

If the artificial joint is designed as a joint of the lower extremity, provision is made, in one variant, that the resistance is increased for the stance phase, e.g. during walking, with rising temperature. Both the extension resistance and also the flexion resistance can be increased. The increased resistance has the effect that the user is forced to walk more slowly and can therefore introduce less energy into the joint. The joint can thus cool down, such that it can be operated within the permissible operating parameters.

In a further variant concerning the use of an artificial joint on a lower extremity, provision is made that, during walking, the flexion resistance is reduced for the swing phase with rising temperature. If the flexion resistance is reduced in or for the swing phase, this has the effect that the joint swings further out. The forwardly moving prosthetic foot thus arrives later at heel strike, as a result of which the user is in turn forced to walk more slowly, which leads to a reduced energy conversion to heat.

The resistance can be changed when a temperature threshold value is reached or exceeded. The resistance can be changed abruptly when a temperature threshold is reached or exceeded, resulting in switching of the resistance value or the resistance values. Provision is advantageously made that the resistance changes continuously with the temperature, after the temperature threshold is reached. How high the temperature threshold value is set depends on the particular design parameters, on the materials used, and on the desired uniformity of the resistance behavior of the prosthesis or orthosis. The resistance in the stance phase, for example, should not be increased to such an extent as to create a situation that compromises safety, e.g. when walking down stairs.

The temperature-induced change of resistance is not the only control parameter of a change of resistance. Instead, provision is made that such a temperature-induced change of resistance superposes a functional change of resistance. An artificial joint, for example a knee joint or an ankle joint, is controlled in a situation-dependent manner via a large number of parameters, such that so-called functional changes of resistance, which take place for example on the basis of the walking speed, the walking situation or the like, are supplemented by the change of resistance resulting from the temperature.

Provision can also be made that a warning signal is output when a temperature threshold value is reached or exceeded, in order to show the user of the prosthesis or orthosis that the joint or the resistance device is in a critical temperature range. The warning signal can be output, as a tactile, visual, or acoustic warning signal. Combinations of the different output possibilities are likewise provided.

The temperature of the resistance device is preferably measured and used as a basis for the control, or other devices can also be subject to the temperature measurement, if the behavior of these devices is temperature-critical. For example, if the control electronics are particularly sensitive to temperature, it is recommended that these be monitored alternatively or in addition to the resistance device and that they be provided with a corresponding temperature sensor. If individual components are temperature-sensitive, for example because of the materials used, it is recommended to provide a measurement device at the corresponding locations in order to be able to obtain corresponding temperature signals.

An adjustment device can be provided, via which the degree of the change of resistance is changed. For example, on the basis of detected data, for example the weight of the user of the orthosis or prosthesis or the determined axial force when the foot is set down, it is possible to identify that an over proportionality high resistance change has to take place. It is likewise possible to provide a manual adjustment device for adapting the respective change of resistance, such that a greater or lesser change of resistance can take place depending on adjusted or determined data.

In the appliance according to the invention for carrying out the method as described above, provision is made that an adjustable resistance device, which is arranged between two mutually articulated components of an artificial orthotic or prosthetic joint, and a control device and sensors, which detect status information of the appliance, are present. At least one temperature sensor and an adjustment device are provided, and a temperature-dependent change of resistance can be activated and/or deactivated via the adjustment device. It is thus possible to perform an optional temperature-controlled change of resistance and to consciously activate or deactivate this special function or auxiliary function of, for example, a knee control method.

An illustrative embodiment of the invention is explained in more detail below with reference to the attached figures, in which:

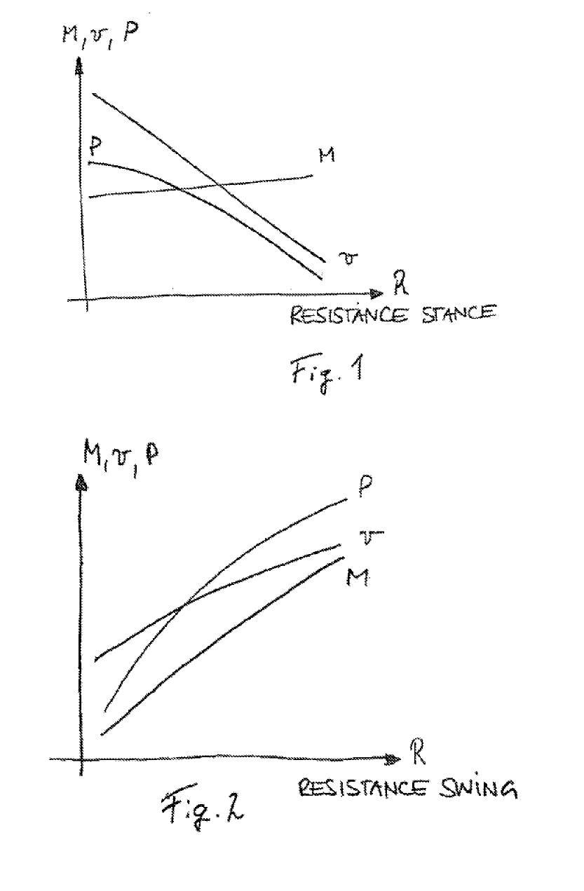

FIG. 1 shows the behavior of variables with increasing resistance in the stance phase;

FIG. 2 shows the behavior of variables with decreasing resistance in the swing phase;

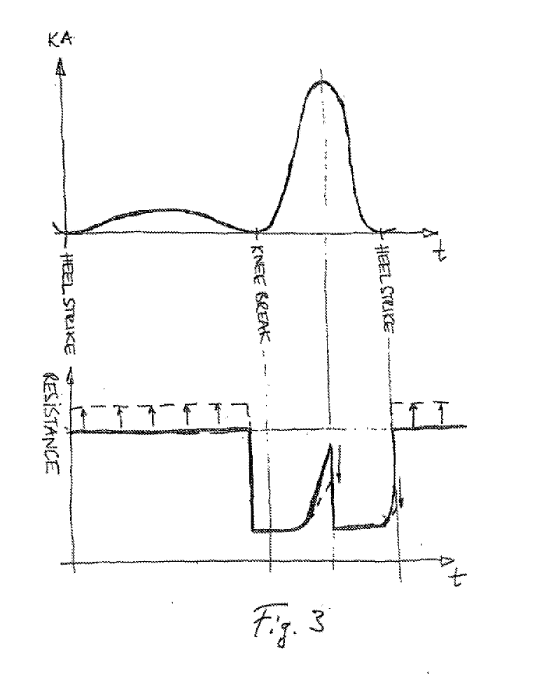

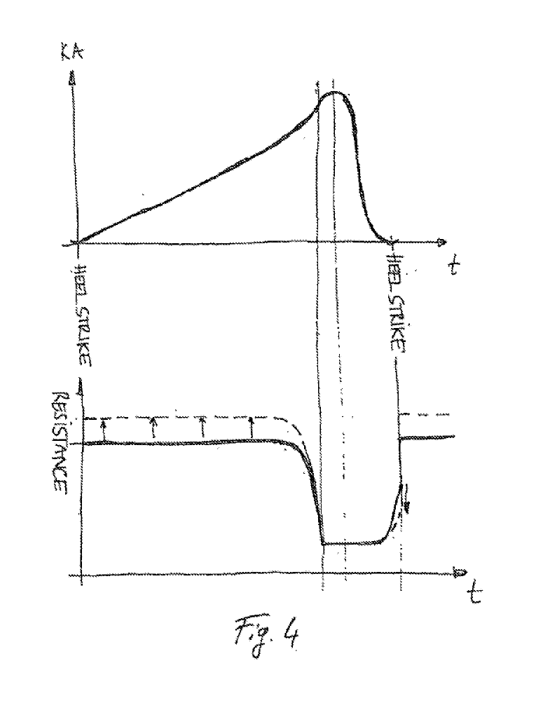

FIG. 3 shows the knee angle profile and resistance curve when walking on the flat; and

FIG. 4 shows the knee angle profile and resistance curve when walking on an incline.

FIG. 1 is a diagram in which, by way of example, the dependency of the variables knee moment M, power P and velocity v is plotted over resistance R in the stance phase of a prosthetic knee joint. The prosthetic knee joint is provided with a resistance device and an actuator, via which actuator the resistance against flexion and/or extension can be changed. In addition to a prosthesis, a correspondingly equipped orthosis can also be used, and the field of use also covers other joint devices, for example hip joints or ankle joints. In the resistance device, the mechanical energy is generally converted to thermal energy, in order to brake the movement of lower leg part relative to a thigh part. The same applies for other joints.

The temperature of the resistance device is dependent on how great the power P is that is applied during the stance phase. The power P is dependent on the active knee moment M, and on the velocity v at which the knee joint is bent. This velocity is in turn dependent on the resistance R with which the resistance device (not shown) opposes the respective movement in the stance phase. If, particularly when walking down ramps or stairs, where the resistance device is typically heated most strongly, the flexion resistance is increased in the stance phase after heel strike and, thereafter, the extension resistance is increased in a starting extension movement, the movement velocity of the joint components with respect to one another decreases, as also does the movement velocity of the resistance device. On account of the over-proportionally stronger decrease in the velocity v, the power P decreases during the stance phase despite an increasing moment M, such that the energy to be converted decreases in accordance with the decreasing power P. Accordingly, with constant cooling, the temperature of the resistance device or of those components whose temperature is monitored decreases.

FIG. 2 shows the correlation of the described variables to the resistance R in the swing phase. When the resistance R decreases during the swing phase, the velocity v, the knee moment M and therefore also the applied power P decrease, such that the energy to be converted is reduced. Accordingly, the temperature of the resistance device decreases as the swing phase resistance fails.

FIG. 3 shows, in the upper diagram, the knee angle KA over time t, beginning with the so-called heel strike, which generally occurs with an extended knee joint. As the foot is set down, a slight flexion of the knee joint, the so-called stance phase flexion, takes place in order to cushion the set-down of the foot and the heel strike. After the foot has been set down fully, the knee is completely extended until the so-called knee break, in which the knee joint is bent in order to move the knee joint forward and perform a rolling movement across the front foot. Starting from the knee break, the knee angle KA increases as far as the maximum angle in the swing phase, in order thereafter to return to an extended position, after the bent knee and the prosthetic foot have been brought forward, and then set the foot down again with the heel. This knee angle profile is typical for walking on the flat.

In the lower graph, the resistance R is plotted over time, corresponding to the matching knee angle. Whether an extension resistance or flexion resistance is present depends on the knee angle profile, with the flexion resistance being active at an increasing knee angle, and the extension resistance being active at a decreasing knee angle. After the heel strike, a relatively high flexion resistance is present; after the movement reversal, there is a high extension resistance. At knee break, the resistance is reduced in order to make it easier to bend the knee and bring it forward. Walking is made easier in this way. After the decrease in resistance at knee break, the resistance is kept at the low level over part of the swing phase, in order to make it easier to swing the prosthetic foot back. To ensure that the swing movement, is not too great, the flexion resistance is increased before the knee angle maximum is reached, and the extension resistance is reduced to the low level of the swing phase flexion after the knee angle maximum and the movement reversal have been reached. The reduction in the extension resistance is also maintained over the extension movement, in the swing phase, until shortly before the heel strike. Shortly before the complete extension is reached, the resistance is increased again in order to avoid a hard strike on the extension limit stop. In order to obtain sufficient safety against uncontrolled buckling when the prosthetic foot is set down, the flexion resistance is also at a high level.

If the flexion resistance is now increased, as is indicated by the broken line, this slows down the knee angle velocity and therefore also the walking action of the prosthesis user. After the heel strike, there is only a comparatively slight bending in the stance phase flexion, and a slow extension, which lessens the dissipated energy. The increase in the flexion resistance, before the knee angle maximum is reached, takes place in a less pronounced way than in the standard damping, as is indicated by the downwardly directed arrow. The lower leg and thus the prosthetic foot swing further out, such that there is a longer period of time between the heel strikes. The reduction in the flexion resistance in the swing phase flexion also leads to a reduction in the walking speed.

At the end of the swing phase extension, i.e. shortly before set-down of the foot and heel strike, the extension resistance is reduced compared to the standard level. Provision is therefore made that the extension resistance is reduced, such that the lower leg part reaches extension more quickly, with the result that the power P, and therefore the energy to be dissipated, decreases. During the stance phase between heel strike and knee break, it is possible for both the flexion resistance and also the extension resistance to be increased, in order to lessen bending during the stance phase and thereby reduce the dissipated energy.

The upper part of FIG. 4 shows the knee angle profile when walking on a ramp, in this case on a downward ramp. The he strike is followed by a continuous increase in the knee angle KA as far as the knee angle maximum. This angle profile arises when walking down a ramp. The knee takes up the weight of the wearer, slowly gives way and thus relieves the load on the contralateral limb. After the knee angle maximum has been reached, the knee and lower leg are brought quickly forward until complete extension, which occurs with the renewed heel strike. The flexion resistance remains at a constantly high level over a wide course, until it is then lowered in order to permit a wide bending of the knee and therefore a lifting of the prosthetic foot and a backward swing. This backward swing takes place after the minimum of the resistance has been reached and until the knee angle maximum is reached. The extension resistance is then maintained at a low resistance level, until being increased again shortly before the foot is set down.

If high temperatures are now present in the resistance device, the resistances in the stance phase are increased in order to ensure a slow walking speed and a slow bending. After the swing phase has been reached and the prosthetic foot has been brought forward, the extension resistance is also reduced, which likewise leads to a reduction in the energy to be dissipated.

1. A method for controlling an artificial orthotic or prosthetic joint with a resistance device to which at least one actuator is assigned, via which actuator the flexion and/or extension resistance is changed depending on sensor data, characterized in that the resistance is changed depending on at least one measured temperature signal.

2. The method as claimed in claim 1, characterized, in that the artificial joint is designed as a joint of a lower extremity, and the resistance is increased during the stance phase with rising temperature.

3. The method as claimed in claim 1 or 2, characterized in that the artificial joint is designed as a joint of a lower extremity, and the flexion resistance is reduced during the swing phase with rising temperature.

4. The method as claimed in one of the preceding claims, characterized in that the resistance is changed when a temperature threshold value is reached or exceeded.

5. The method as claimed in one of the preceding claims, characterized in that the resistance is changed continuously with the changing temperature.

6. The method as claimed in one of the preceding claims, characterized in that the temperature-induced change of resistance superposes a functional change of resistance.

7. The method as claimed in one of the preceding claims, characterized in chat a warning signal is output when a temperature threshold value is reached or exceeded.

8. The method as claimed in one of the preceding claims, characterized in that the temperature of the resistance device is measured and used as a basis for the control.

9. The method as claimed in one of the preceding claims, characterized in that an adjustment device is provided, via which the degree of the change of resistance is changed.

10. An appliance for carrying out she method as claimed in one of the preceding claims, with an adjustable resistance device, which is arranged between two mutually articulated components of an artificial orthotic or prosthetic joint, and with a control device and sensors, which detect status information of the appliance, characterized in that at least one temperature sensor and an adjustment device are provided, and in that a temperature-dependent change of resistance can be activated and/or deactivated via the adjustment device.
